(19) United States
(12) Patent Application Publication
(10) Pub. No.: US 2026/0265652 A1
(43) Pub. Date: Sep. 10, 2026

(54) FIXED BED BIOFILM REACTOR MEDIA BLOCK

(71) Applicant: Advanced Drainage Systems, Inc., Hilliard, OH (US)

(72) Inventors: Cal PITRUZZELLO, Middlefield, CT (US); Paul HOLBROOK, Old Saybrook, CT (US)

(73) Assignee: Advanced Drainage Systems, Inc., Hilliard, OH (US)

(21) Appl. No.: 19/557,755

(22) Filed: Mar. 5, 2026

Related U.S. Application Data
(60) Provisional application No. 63/768,395, filed on Mar. 7, 2025.

(51) Int. Cl. C12M 1/12

(52) U.S. Cl. CPC C12M 23/04; C12M 25/18

(57) ABSTRACT

Consistent with disclosed embodiments, systems, methods, and apparatuses for a foldable sheet configured to form a media block are disclosed. The foldable sheet may have a first media unit, which may include a first layer having an upper portion comprising a plurality of pockets and a lower portion comprising a plurality of protrusions. The first media unit may further comprise a second layer having an upper portion comprising a plurality of protrusions and a lower portion comprising a plurality of pockets. The foldable sheet may further comprise a hinge connecting the first layer to the second layer, and a flat portion connecting the second layer of the first media unit to a first layer of a second media unit.

### CROSS-REFERENCE TO RELATED APPLICATIONS

This application claims the benefit of priority of U.S. Provisional Patent Application No. 63/768,395, filed March 7, 2025, the contents of which are incorporated herein by reference in their entirety

### TECHNICAL FIELD

This disclosure relates generally to systems, methods, and apparatuses for forming a fixed bed biofilm reactor (“FBBR”) media block, and more particularly, to forming an FBBR media block from a single sheet or a folded sheet.

### BACKGROUND

Wastewater from residential or commercial buildings may contain pollutants and contaminants, such as ammonia and nitrate, biochemical oxygen demand (BOD), and total suspended solids (TSS). Wastewater treatment units may include biological treatment systems to treat such wastewater with high levels of organic contamination. For example, FBBR may be used to remove organic contaminants and pollutants from wastewater. FBBRs may include one or more media blocks. A layer of biofilm may attach to and grow on the FBBR media blocks and may remove solids and organic contaminants from wastewater flowing through the media blocks. FBBR systems may provide a high rate of pollutant removal within a small area of a wastewater treatment unit.

However, solutions are needed to provide improved processes for forming FBBR media blocks. Current techniques for forming FBBR media blocks may include manufacturing individual sheets of the media block and attaching such sheets together through adhesives, mechanical joints, or heat staking. However, such techniques can be time consuming and cost ineffective. Solutions should be provided that allow for faster and more cost-effective formation of FBBR media blocks. Such solutions should include forming one continuous sheet that may be folded to form an FBBR media block. Such solutions should further include forming identical single sheets that can be stacked to form an FBBR media block.

### SUMMARY

Consistent with disclosed embodiments, systems, methods, and apparatuses for a foldable sheet configured to form a media block as disclosed. The foldable sheet may have a first media unit, which may include a first layer, which may include an upper portion comprising a plurality of pockets, and a lower portion comprising a plurality of protrusions. The first media unit may further comprise a second layer, which may comprise an upper portion comprising a plurality of protrusions, and a lower portion comprising a plurality of pockets. The foldable sheet may further comprise a hinge connecting the first layer to the second layer, and the flat portion connecting the second layer of the first media unit to a first layer of a second media unit, wherein the second media unit may be identical to the first media unit.

In some embodiments the foldable sheet may comprise a plurality of connected media units. In some embodiments, the plurality of protrusions may each comprise a standoff and a dowel. In some embodiments, the standoff may be configured to create a gap between the first layer and the second layer of the first media unit when the foldable sheet is in a folded configuration. In some embodiments, the standoff may comprise a base and the dowel may extend from the standoff. In some embodiments, the plurality of pockets of the first layer may be configured to engage with the plurality of protrusions of the second layer. In some embodiments, the hinge may comprise a living hinge. In some embodiments, the hinge may be configured to fold the first layer and the second layer of the first media unit 180 degrees. In some embodiments, the first layer and the second layer may comprise a plurality of ribs. In some embodiments, a plurality of first layers and a plurality of second layers of a plurality of media units may be configured to be folded to form the media block.

The disclosed embodiments further include systems, methods, and apparatuses for a sheet configured to form a media block. The sheet may comprise a plurality of corrugations, a plurality of pockets on a first side of the sheet, wherein the plurality of pockets may be formed in at least one of the plurality of corrugations, and a plurality of dowels on a second side of the sheet.

In some embodiments, the plurality of dowels may extend outwardly from the second side of the sheet. In some embodiments, the plurality of dowels of the sheet may be configured to engage with a plurality of pockets of a second sheet. In some embodiments, the sheet may be configured to be stacked with a plurality of identical sheets to form the media block. In some embodiments, the sheet may be formed by thermoforming. In some embodiments, the sheet may be formed by injection molding. In some embodiments, the sheet may be configured to be stacked with a second identical sheet, wherein the second identical sheet may be rotated 180 degrees about a z-axis from the sheet. In some embodiments, the plurality of dowels of the sheet may be configured to snap fit with a plurality of pockets of an identical second sheet. In some embodiments, the plurality of dowels of the sheet may be configured to form a friction fit with a plurality of pockets of an identical second sheet. In some embodiments, the plurality of corrugations may comprise a plurality of ribs configured to facilitate biological growth along the plurality of ribs.

Additional features and advantages of the disclosed embodiments will be set forth in part in the description that follows, and in part will be obvious from the description, or may be learned by practice of the disclosed embodiments. The features and advantages of the disclosed embodiments will be realized and attained by the elements and combinations particularly pointed out in the appended claims.

It is to be understood that both the foregoing general description and the following detailed description are examples and explanatory only and are not restrictive of the disclosed embodiments as claimed.

### BRIEF DESCRIPTION OF THE DRAWINGS

The accompanying drawings constitute a part of this specification. The drawings illustrate several embodiments of the present disclosure and, together with the description, serve to explain the principles of the disclosed embodiments as set forth in the accompanying claims.

FIG. 1 depicts a foldable sheet for forming an FBBR media block, according to disclosed embodiments.

FIG. 2 depicts an enlarged view of the folding sheet of FIG. 1, according to disclosed embodiments.

FIG. 3 depicts an isometric view of an assembled FBBR media block, according to disclosed embodiments.

FIG. 4 depicts a top view of an assembled FBBR media block, according to disclosed embodiments.

FIG. 5 depicts a section cut of an assembled FBBR media block, according to disclosed embodiments.

FIG. 6A depicts a top side of a single sheet for forming an FBBR media block, according to disclosed embodiments.

FIG. 6B depicts a bottom side of a single sheet for forming an FBBR media block, according to disclosed embodiments.

FIGS. 7A-7C depict a process for assembling an FBBR media block, according to disclosed embodiments.

FIG. 8 depicts a section cut of an assembled FBBR media block, according to disclosed embodiments.

FIG. 9 depicts an assembled FBBR media block, according to disclosed embodiments.

FIG. 10A depicts a first layer for forming a lamella clarifier, according to disclosed embodiments.

FIG. 10B depicts a second layer for forming a lamella clarifier, according to disclosed embodiments.

FIG. 11 depicts the first layer of FIG. 10A and the second layer of FIG. 10B stacked together for forming a lamella clarifier, according to disclosed embodiments.

### DETAILED DESCRIPTION

Examples of embodiments of the present disclosure are described with reference to the accompanying drawings. In the figures, which are not necessarily drawn to scale, wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items or meant to be limited to only the listed item or items. It should also be noted that as used in the present disclosure and in the appended claims, the singular forms “a,” “an,” and “the” include plural references unless the context clearly dictates otherwise.

The disclosed embodiments improve deficiencies in existing methods and techniques for forming FBBR media blocks. The disclosed embodiments provide a fast and cost-effective method for forming FBBR media blocks. For example, the disclosed embodiments include methods for forming an FBBR media block from a continuous, foldable sheet. The disclosed embodiments also include forming identical single sheets that can be stacked to form an FBBR media block.

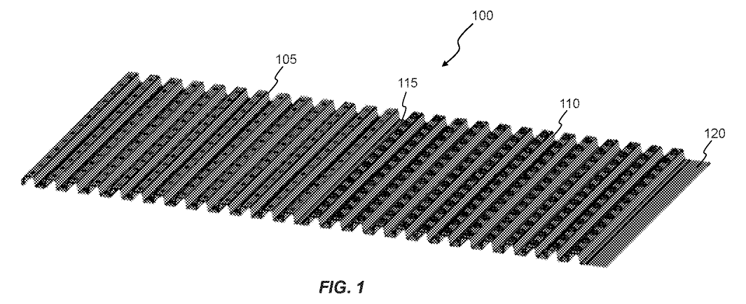

FIG. 1 depicts foldable sheet 100, which may be used to form an FBBR media block according to disclosed embodiments. Foldable sheet 100 may include first layer 105 and second layer 110 separated by hinge 115. In some embodiments, foldable sheet 100 may be formed from polypropylene (“PP”), polyethylene (“PE”), polystyrene, polyvinyl chloride (“PVC”), polyethylene terephthalate (“PET”), or any other plastic or non-plastic material. In some embodiments, foldable sheet 100 may be thermoformed.

First layer 105 and second layer 110 may comprise a plurality of pockets and protrusions, as disclosed herein, that may be configured to mate with each other when foldable sheet 100 is assembled into a folded configuration. For example, foldable sheet 100 may be configured to be folded 180 degrees such that first layer 105 and second layer 110 are in contact with each other, as disclosed herein. Hinge 115 may facilitate the folding of first layer 105 and second layer 110 towards each other. In some embodiments, hinge 115 may comprise a living hinge, which may include a flexible hinge made from the same material as first layer 105 and second layer 110. For example, hinge 115 may be integral to first layer 105 and second layer 110.

Foldable sheet 100 may further include flat portion 120. Flat portion 120 may comprise a section between repeating layers of foldable sheet 100. For example, although FIG. 1 depicts first layer 105 and second layer 110 separated by hinge 115, the pattern of first layer 105, hinge 115, second layer 110, flat portion 120 may repeat after flat portion 120 depicted in FIG. 1. Flat portion 120 may provide space between the repeated pattern of first layer 105, hinge 115, and second layer 110. Flat portion 120 may further facilitate folding of foldable sheet 100 after foldable sheet 100 has been fully formed.

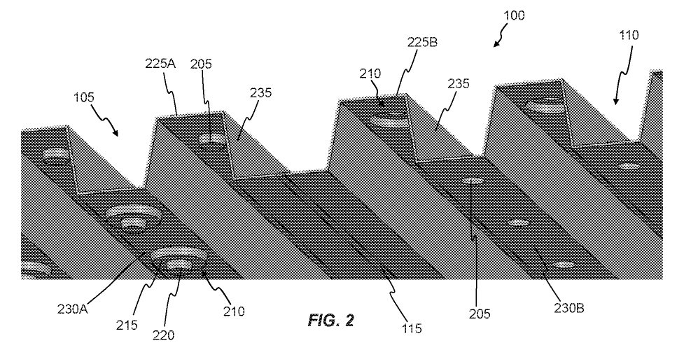

FIG. 2 depicts an enlarged view of foldable sheet 100. As depicted in FIG. 2, first layer 105 may include upper portion 225A and lower portion 230A and second layer 110 may include upper portion 225B and lower portion 230B. Each of upper portion 225A-B and lower portion 230A-B may be separated by walls 235 extending between upper portion 225A-B and lower portion 230A-B. In some embodiments, walls 235 may extend upwardly from lower portion 230A-B at an angle. In other embodiments, walls 235 may extend perpendicularly upwardly from lower portion 230A-B. Although FIG. 2 depicts upper portions 225A-B, lower portions 230A-B, and walls 235 as substantially flat surfaces, each of upper portions 225A-B, lower portions 230A-B, and walls 235 may include ribbed surfaces. The ribs of the ribbed surfaces may be any size, shape, and configuration to aid in and facilitate biological growth when foldable sheet 100 is formed into an FBBR media block and placed within a wastewater treatment system.

As depicted in FIG. 2, upper portion 225A of first layer 105 and lower portion 230B of second layer 110 may include a plurality of pockets 205. Pockets 205 may include openings through upper portion 225A of first layer 105 and lower portion 230B of second layer 110. Although FIG. 2 depicts pockets 205 as circular in shape, pockets 205 may be elliptical, rectangular, square, or any other shape.

Lower surface 230A of first layer 105 and upper surface 225B of second layer 110 may include protrusions 210. Protrusions 210 may comprise standoff 215 and dowel 220. Standoff 215 may comprise a wider base from which dowel 220 may extend. For example, a diameter of standoff 215 may be greater than a diameter of dowel 220. Further, the diameter of standoff 215 may be greater than a diameter of pocket 205. Although FIG. 2 depicts protrusions 210 as circular in shape, protrusions 210 may be elliptical, rectangular, square, or any other shape. The size and shape of dowel 220 may correspond to the size and shape of pocket 205. Accordingly, when sheet 100 is folded such that first layer 105 is in contact with second layer 110, dowels 220 may interlock with pockets 205. The interlocking of dowels 220 and pockets 205 may secure first layer 105 to second layer 110. When sheet 100 is in a folded configuration, standoff 215 may create a gap between lower portion 230A of first layer 105 and lower portion 230B of second layer 110, as disclosed herein. The gap between lower portion 230A of first layer 105 and lower portion 230B of second layer 110 may increase the surface area along lower portion 230A and lower portion 230B that may be utilized for biological growth when foldable sheet 100 is formed into an FBBR media block and placed in a wastewater treatment system.

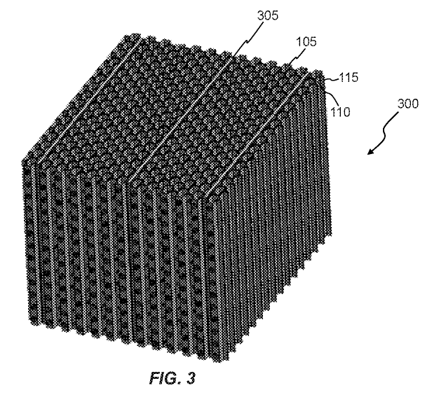

FIG. 3 depicts an assembled FBBR media block 300, which may be formed from foldable sheet 100. For example, foldable sheet 100, as depicted in FIG. 1, may be formed from a plurality of connected, repeating media units, which may include first layer 105, hinge 115, second layer 110, and flat portion 120. The repeating media units of foldable sheet 100 may be thermoformed in one, continuous sheet. For example, the flat portion may connect a second layer of a first media unit to a first layer of a second media unit. Foldable sheet 100 may be formed from as many repeating media units as required to form an FBBR media block 300 of any size. For example, as depicted in FIG. 3, foldable sheet 100 may include sixteen repeating, connected media units, including first layer 105, hinge 115, second layer 110, and flat portion 120. In other embodiments, foldable sheet 100 may include more or fewer repeating, connected media units. After foldable sheet 100 has been thermoformed as one continuous sheet, first layer 105 may be folded 180 degrees toward second layer 110 by hinge 115. Second layer 110 may be folded 180 degrees toward the next repeating first layer by flat portion 120. This pattern of repetitive folding of each layer may be continued until all the first layers 105 and second layers 110 have been folded to form FBBR media block 300.

As depicted in FIG. 3, FBBR media block 300 may be formed from a plurality of first layers 105 and second layers 110 folded together by hinge 115. The pockets and protrusions of first layer 105 and second layer 110 (as depicted in FIG. 2) may secure the plurality of first layers 105 to the plurality of second layers 110. Bands 305 may be applied around FBBR media block 300 to further secure FBBR media block 300 in its assembled configuration. Although FIG. 3 depicts three bands 305, FBBR media block 300 may be secured by any number of bands 305. When FBBR media block 300 is installed within a wastewater treatment system, biological growth may grow across the surfaces of FBBR media block 300. The biological growth may facilitate treatment and filtration of a flow of wastewater that may flow through FBBR media block 300 when FBBR media block 300 is installed within a wastewater treatment system.

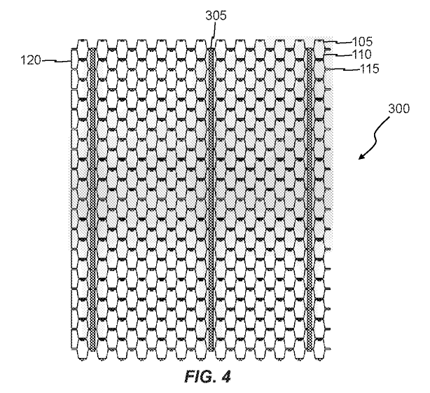

FIG. 4 depicts a top view of an assembled FBBR media block 300. As depicted in FIG. 4, FBBR media block 300 may be formed by folding a plurality of first layers 105 and second layers 110 by hinge 115 and flat portion 120. For example, as disclosed herein, FBBR media block 300 may be formed by folding the repeating layers of one, continuous sheet, such as foldable sheet 100. Band 305 may be applied around assembled FBBR media block 300 to further secure FBBR media block 300 in its assembled configuration.

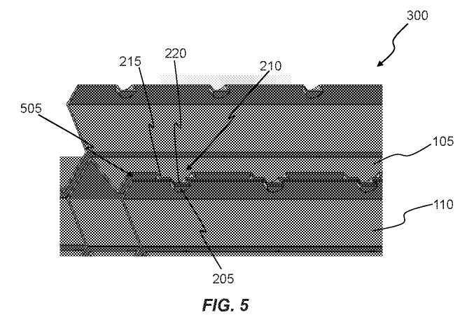

FIG. 5 depicts an enlarged section view of FBBR media block 300. As depicted in FIG. 5, first layer 105 and second layer 110 may be interconnected by pocket 205 and dowel 220 of protrusion 210. Standoff 215 of protrusion 210 may form gap 505 between first layer 105 and second layer 110 of FBBR media block 300. Gap 505 may allow for biological growth between first layer 105 and second layer 110 of FBBR media block 300.

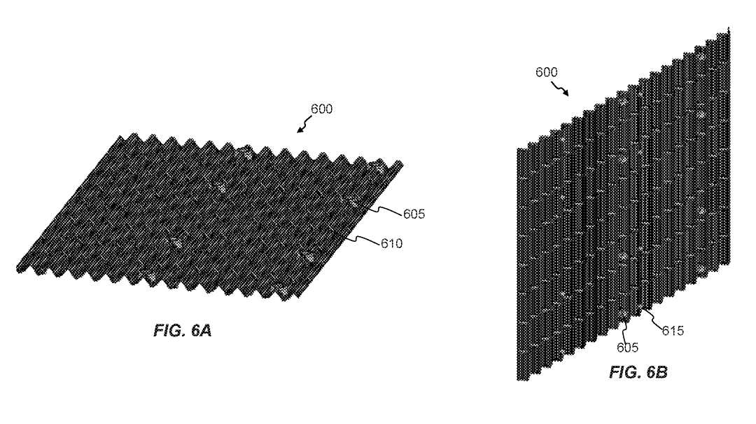

FIG. 6A depicts a top side of sheet 600 and FIG. 6B depicts a bottom side of sheet 600, which may be used to form an FBBR media block. In some embodiments, sheet 600 may be formed from polypropylene (“PP”), polyethylene (“PE”), polystyrene, polyvinyl chloride (“PVC”), polyethylene terephthalate (“PET”), or any other plastic or non-plastic material. In some embodiments, sheet 600 may be thermoformed or injection molded. As depicted in FIG. 6A, sheet 100 may include a plurality of corrugations 610. Corrugations 610 may include a plurality of ribs, which may aid in and facilitate biological growth along sheet 600.

Sheet 600 may further include pockets 605. Pockets 605 may comprise openings through corrugations 610. For example, a plurality of pockets 605 may be spaced along at least one row of corrugations of sheet 600. Sheet 600 may include a plurality of pockets extending along two rows of corrugations, as depicted in FIGS. 6A and 6B. In other embodiments, sheet 600 may include more or fewer rows of pockets 605. Although FIGS. 6A and 6B depict pockets 605 as circular in shape, pockets 605 may be elliptical, rectangular, square, or any other shape. As depicted in FIG. 6B, sheet 600 may further include dowels 615. Dowels 615 may include projections from the bottom surface of sheet 600. For example, dowels 615 may project from a peak of a row of corrugations 610. Sheet 600 may include two rows of dowels 615, as depicted in FIG. 6B. In other embodiments, sheet 600 may include more or fewer rows of dowels 615. In some embodiments, dowels 615 may be circular in shape. In other embodiments, dowels 615 may be elliptical, square, rectangular, or any other shape. The size and shape of dowels 615 may correspond to the size and shape of pockets 605, such that dowels 615 of a first sheet may be interconnected with pockets 605 of a second sheet.

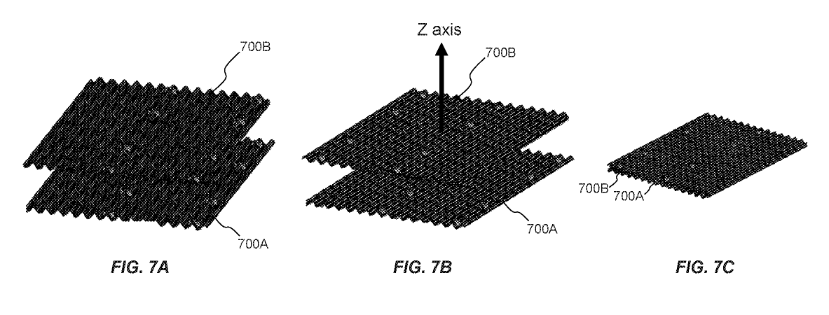

FIGS. 7A-7C depict a method of connecting first sheet 700A to second sheet 700B. First sheet 700A and second sheet 700B may correspond to sheet 600, as disclosed herein with respect to FIGS. 6A and 6B. First sheet 700A and second sheet 700B may be identical in size, shape, and configuration, as depicted in FIG. 7A. In some embodiments, when first sheet 700A and second sheet 700B are thermoformed or injection molded, they may be oriented in the same direction, as depicted in FIG. 7A. In other embodiments, second sheet 700B may be rotated 180 degrees about the Z-axis, as depicted in FIG. 7B. Rotating second sheet 700B 180 degrees may allow the dowels of second sheet 700B to align with the pockets of first sheet 700A, such that the dowels of second sheet 700B may interconnect with the pockets of first sheet 700A. As depicted in FIG. 7C, second sheet 700B may be placed on first sheet 700A, such that the dowels of second sheet 700B interconnect with the pockets of first sheet 700A. Interconnecting the dowels of second sheet 700B with the pockets of first sheet 700A may provide a secure connection between first sheet 700A and second sheet 700B. Although FIGS. 7A-7C depict two sheets stacked together, any number of sheets may be stacked together by following the pattern of rotating adjacent sheets 180 degrees.

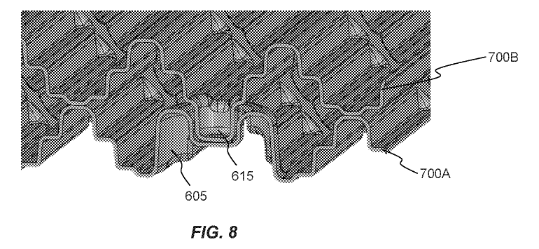

FIG. 8 depicts an enlarged section view of first sheet 700A and second sheet 700B in a connected configuration. As depicted in FIG. 8, dowel 615 of second sheet 700B may be interconnected with pocket 605 of first sheet 700A. The size, shape, and configuration of dowel 615 may correspond to the size, shape, and configuration of pocket 605, such that dowel 615 may rest within pocket 605. In some embodiments, the connection between dowel 615 and pocket 605 may include a snap fit or a friction fit connection. Connecting dowel 615 and pocket 605 may secure first sheet 700A to second sheet 700B when forming an assembled FBBR media block.

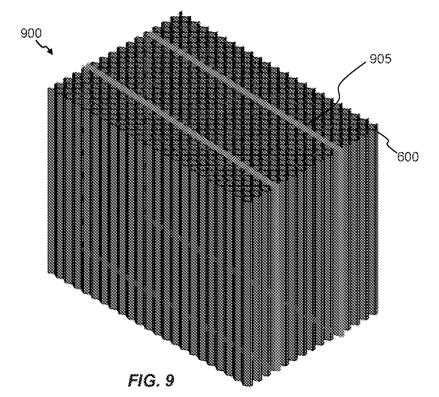

FIG. 9 depicts an assembled FBBR media block 900 formed from a plurality of stacked sheets 600. Sheets 600 may be stacked as described herein with respect to FIG. 7A-7C to form a complete, assembled FBBR media block 900. Any number of sheets may be stacked together to form an FBBR media block of any size. FBBR media block 900 may include straps 905. Straps 905 may be placed around FBBR media block 900 to secure each of the sheets together to maintain the assembled configuration. When FBBR media block 900 is placed in a wastewater treatment system, biological growth may attach to and grow on the surfaces of FBBR media block 900. The biological growth may treat and filter a flow of wastewater through FBBR media block 900 when FBBR media block 900 is installed within a wastewater treatment system.

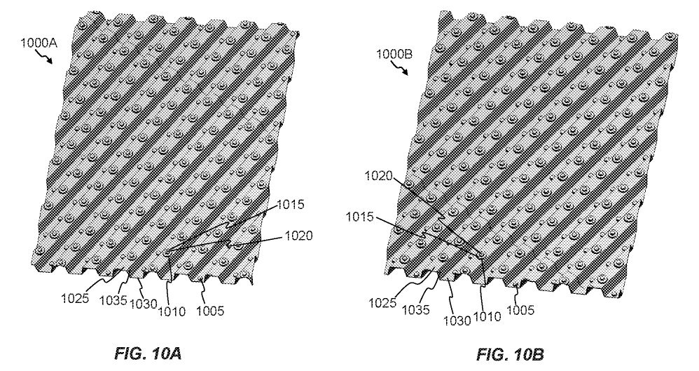

FIG. 10A depicts first layer 1000A and FIG. 10B depicts second layer 1000B, which may be stacked to form a lamella clarifier. In some embodiments, first layer 1000A and second layer 1000B may be formed from polypropylene (“PP”), polyethylene (“PE”), polystyrene, polyvinyl chloride (“PVC”), polyethylene terephthalate (“PET”), or any other plastic or non-plastic material. In some embodiments, first layer 1000A and second layer 1000B may be thermoformed or injection molded.

Both first layer 1000A and second layer 1000B may include upper portions 1025 and lower portions 1030. Upper portions 1025 and lower portions 1030 may be separated by walls 1035 extending between upper portions 1025 and lower portions 1030. In some embodiments, walls 1035 may extend upwardly from lower portions 1030 at an angle. In other embodiments, walls 1035 may extend perpendicularly upwardly from lower portions 1030.

Upper portions 1025 and lower portions 1030 of first layer 1000A may be offset from upper portions 1025 and lower portions 1030 of second layer 1000B. For example, an offset between upper portions 1025 and lower portions 1030 of first layer 1000A and upper portions 1025 and lower portions 1030 of second layer 1000B may allow upper portions 1025 of second layer 1000B to engage with lower portions 1030 of first layer 1000A when first layer 1000A is stacked on second layer 1000B, as disclosed herein.

As depicted in FIG. 10A and FIG. 10B, lower portions 1030 of first layer 1000A and second layer 1000B may include a plurality of pockets 1005. Pockets 1005 may correspond to pockets 205, as described herein with respect to FIG. 2. For example, pockets 1005 may include openings through lower portion 1030 of first layer 1000A and second layer 1000B. Although FIG. 10A and FIG. 10B depict pockets 1005 as circular in shape, pockets 1005 may be elliptical, rectangular, square, or any other shape.

Upper surface 1025 of first layer 1000A and second layer 1000B may include protrusions 1010. Protrusions 1010 may correspond to protrusions 210, as disclosed herein with respect to FIG. 2. For example, protrusions 1010 may comprise standoff 1015 and dowel 1020. Standoff 1015 may comprise a wider base from which dowel 1020 may extend. For example, a diameter of standoff 1015 may be greater than a diameter of dowel 1020. Further, the diameter of standoff 1015 may be greater than a diameter of pocket 1005. Although FIG. 10A and FIG. 10B depict protrusions 1010 as circular in shape, protrusions 1010 may be elliptical, rectangular, square, or any other shape. The size and shape of dowel 1020 may correspond to the size and shape of pocket 1005. Accordingly, when first layer 1000A is stacked on second layer 1000B, dowels 1020 of second layer 1000B may interlock with pockets 1005 of first layer 1000A. The interlocking of dowels 1020 and pockets 1005 may secure first layer 1000A to second layer 1000B.

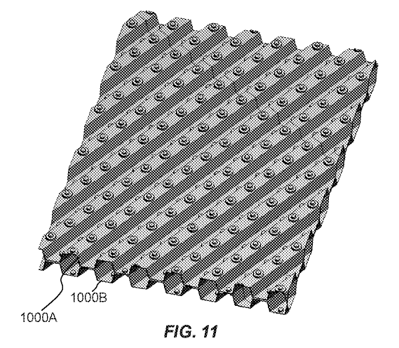

FIG. 11 depicts first layer 1000A stacked on second layer 1000B to form a lamella clarifier. Although FIG. 11 depicts one first layer 1000A and one second layer 1000B, a lamella clarifier may be formed from any number of stacked alternating first layers 1000A and second layers 1000B. For example, in some embodiments, a lamella clarifier having 2, 3, 4, 5, 6, 7, 8, 9, 10, or more first layers 1000A and 2, 3, 4, 5, 6, 7, 8, 9, 10, or more second layers 1000B may be formed. The offset pattern of upper portions 1025 and lower portions 1030 of first layer 1000A and second layer 1000B may allow upper portions 1025 of second layer 1000B to engage with lower portions 1030 of first layer 1000A. A plurality of angled channels may be formed by the stacked first layer 1000A and second layer 1000B through which wastewater may flow when the lamella clarifier is installed within a wastewater treatment system.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps and/or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and, accordingly, all suitable modifications and equivalents may be resorted to, falling within the scope of the disclosure.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A foldable sheet configured to form a media block, wherein the foldable sheet comprises:
a first media unit, comprising:
a first layer, comprising:
an upper portion comprising a plurality of pockets; and
a lower portion comprising a plurality of protrusions;
a second layer, comprising:
an upper portion comprising a plurality of protrusions; and
a lower portion comprising a plurality of pockets;
a hinge connecting the first layer to the second layer; and
a flat portion connecting the second layer of the first media unit to a first layer of a second media unit, wherein the second media unit is identical to the first media unit.

2. The foldable sheet of claim 1, wherein the foldable sheet comprises a plurality of connected media units.

3. The foldable sheet of claim 1, wherein the plurality of protrusions each comprise a standoff and a dowel.

4. The foldable sheet of claim 3, wherein the standoff is configured to create a gap between the first layer and the second layer of the first media unit when the foldable sheet is in a folded configuration.

5. The foldable sheet of claim 3, wherein the standoff comprises a base and the dowel extends from the standoff.

6. The foldable sheet of claim 1, wherein the plurality of pockets of the first layer are configured to engage with the plurality of protrusions of the second layer.

7. The foldable sheet of claim 1, wherein the hinge comprises a living hinge.

8. The foldable sheet of claim 1, wherein the hinge is configured to fold the first layer and the second layer of the first media unit 180 degrees.

9. The foldable sheet of claim 1, wherein the first layer and the second layer comprise a plurality of ribs.

10. The foldable sheet of claim 1, wherein a plurality of first layers and a plurality of second layers of a plurality of media units are configured to be folded to form the media block.

11. A sheet configured to form a media block, wherein the sheet comprises:
a plurality of corrugations;
a plurality of pockets on a first side of the sheet, wherein the plurality of pockets are formed in at least one of the plurality of corrugations; and
a plurality of dowels on a second side of the sheet.

12. The sheet of claim 11, wherein the plurality of dowels extend outwardly from the second side of the sheet.

13. The sheet of claim 11, wherein the plurality of dowels of the sheet are configured to engage with a plurality of pockets of a second sheet.

14. The sheet of claim 11, wherein the sheet is configured to be stacked with a plurality of identical sheets to form the media block.

15. The sheet of claim 11, wherein the sheet is formed by thermoforming.

16. The sheet of claim 11, wherein the sheet is formed by injection molding.

17. The sheet of claim 11, wherein the sheet is configured to be stacked with a second identical sheet, wherein the second identical sheet is rotated 180 degrees about a z-axis from the sheet.

18. The sheet of claim 11, wherein the plurality of dowels of the sheet are configured to snap fit with a plurality of pockets of an identical second sheet.

19. The sheet of claim 11, wherein the plurality of dowels of the sheet are configured to form a friction fit with a plurality of pockets of an identical second sheet.

20. The sheet of claim 11, wherein the plurality of corrugations comprise a plurality of ribs configured to facilitate biological growth along the plurality of ribs.
